(19) United States
(12) Patent Application Publication
(10) Pub. No.: US 2015/0086514 A1
(43) Pub. Date: Mar. 26, 2015

(54) MESENCHYMAL STEM CELL INJECTION AND PREPARATION METHOD THEREOF, AND APPLICATION THEREOF IN PREPARING DIABETES DRUG

(71) Applicant: Zaimei JIA, Zhangqiu City, Shandong (CN)

(72) Inventors: Yuxiang Huang, Qingdao City (CN); Hong Gao, Qingdao City (CN); Li Wang, Qingdao City (CN); Jianxia Hu, Qingdao City (CN); Xuefeng Zhang, Qingdao City (CN)

(21) Appl. No.: 14/387,173

(22) PCT Filed: Nov. 11, 2013
(86) PCT No.: PCT/CN2013/086828

§ 371 Date: Sep. 22, 2014

(30) Foreign Application Priority Data
Nov. 14, 2012 (CN) 201210458314.7

(51) Int. Cl. A61K 35/28; C12N 5/0775

(52) U.S. Cl. CPC A61K 35/28; C12N 5/0668; C12N 2509/10; C12N 2500/90
USPC 424/93.7; 435/379

(57) ABSTRACT

The present invention provides a mesenchymal stem cell injection, a preparation method thereof, and application thereof in preparing diabetes mellitus drug. Mesenchymal stem cells used by the present invention come from the human umbilical cord and the human placenta. The ingredients of the mesenchymal stem cell injection are: mesenchymal stem cells, human albumin, low molecular weight heparin, a compound amino acid, vitamin C, and a solution medium. The solution medium is a compound electrolyte solution, glucose water, or a normal saline solution.

### TECHNICAL FIELD

The present invention relates to the field of biomedicine, and particularly to mesenchymal stem cell injection, a preparation method thereof, and an application thereof in preparing diabetes drug.

### BACKGROUND

The incidence of diabetes mellitus is higher and higher. According to statistics, there are 150 millions of diabetes mellitus patients globally at present, and experts predict that this number will reach 300 millions by 2025, 75% of which are in developing countries such as India, China, etc. In China, the number of Chinese who suffer from diabetes mellitus is on the rise. The statistics show that the incidence rises to the current 2.5-3.25% from the 1% ten years ago, and the incidence of diabetes mellitus in urban adults approaches 10%.

The diabetes mellitus is classified as Type 1, Type 2, Pregnancy and other types of diabetes mellitus. Type 1 diabetes mellitus (T1 DM) is characterized by damage to islet β cells, wherein Type 1 A is T lymphocyte mediated autoimmune disease. Under the interaction of inheritance susceptible factor and environmental factor, imbalance of immunological regulation in the body occurs gradually. The characteristic is serious insulin secretion obstacle caused by the damage of islet β cells, with the continuous rise of blood glucose as the indication. Because the function of the islet β cells of a T1 DM patient is poor, blood glucose is difficult to be controlled, and severe complications such as infection, diabetic microangiopathy, ketoacidosis and the like easily occur, seriously affecting the growth and development of youngsters.

The United Kingdom Prospective Diabetes Study (UKPDS) shows that when a definite diagnosis is made to a patient for Type 2 diabetes mellitus (T2 DM), the function of his/her islet β cells is decreased by as much as 50%. As prolongation of the course of the disease, the function of his/her islet β cells declines at the rate of 6%-8% per year. The persistence of high blood glucose can directly damage the function of islet β cells, weaken insulin secretion response of the islet β cells to the rising of blood glucose and reduce insulin mediated glucose function, as result the further rising of blood glucose occurred. This vicious circle is a life long battle for each diabetic patient. Therefore, many researchers focus on studying how to protect and/or restore the islet β cells, hopefully to change or extend the natural course of Type 2 diabetes mellitus.

At present, the treatment of diabetes mellitus mainly relies on drugs and insulin injection to control blood glucose and for now there is no cure for diabetes mellitus. At onset of the disease, patients need to be treated with drugs and/or insulin injection for life-long. Also Islet transplantation provides possibility for curing diabetes mellitus, but because islet source is limited and the matching type is difficult, success rate of operations is low, and anti-immunological rejection drugs are needed to be taken for long term after the treatments, which limits this technology from wide application.

Furthermore, the current treatment method primarily using insulin can effectively alleviate symptoms of diabetes mellitus, but cannot completely solve the problem of islet β cells injury. Therefore, looking for a treatment method which to promote regeneration of islet β cells, to protect and repair islet β cells is a great topic in medical community.

Mesenchymal stem cells (MSCs) are important members of the stem cell family, and researchers and medical communities are paying more and more attention to them, because they have features such as potential of multi-directional differentiation, hemopoietic support, promoting stem cells implantation and self-replication and the like. Under specific induction conditions in vivo or in vitro, MSCs can differentiate into many tissue cells such as islet, nerve, blood vessel endothelium, bone, cartilage, muscle, liver, cardiac muscle and the like. In addition, MSCs have low immunogenicity and unique immunomodulatory effects which can avoid immune recognition and inhibit immune response. MSCs derived from placenta and umbilical cord have features such as great potential of differentiation, stronger multiplication capacity, lower immunogenicity comparing with MSCs from other sourcese, convenience of obtaining materials, absence of amoral ethics restriction problems, facilitation of industrialized preparation and the like. Due to the above biological characteristics, MSCs can be used as cell therapy for tissue repair, suppression of immune rejection response and metabolic diseases.

To sum up, the current treatment of diabetes mellitus is mainly to control blood glucose, and cannot fundamentally cure diabetes mellitus. The patients need to take drugs for life-long. The toxicity and side effects of the drugs also pose great impacts on the patients' life. Furthermore, with the progress of diabetes mellitus cource, if the blood glucose is not properly controlled, there will be complications related to diabetes mellitus soon, such as diabetes mellitus retinopathy, diabetes mellitus nephropathy, diabetes mellitus peripheral nerve pathology, or even diabetes mellitus foot and other severe complications. The current treatment with drugs and/or insulin cannot well avoid these situations, repair islet β cells and have them regenerated, restore endogenous insulin secretion.

The shortcomings of the prior art are: the current treatment of diabetes mellitus mainly depends on diet control, cinesiotherapy, and combined treatment of drugs and insulin. These therapies are applied in combination, which can control blood glucose stable during short time, but the blood glucose is lowered just by external drugs, which cannot effectively repair functions of damaged islet, prevent disease progression and cannot fundamentally cure the diabetes mellitus. Many hypoglycemic drugs have very severe side effects, such as: severe hypoglycemia, diarrhea, function damage of liver and kidney etc.

### SUMMARY OF THE INVENTION

Aiming to the above mentioned deficiencies and shortcomings of the treatment for diabetes mellitus in the prior art, the present invention provides mesenchymal stem cell injection, a preparation method thereof, and an application thereof in preparing diabetes mellitus drug. With the technical solution of the present invention, the present invention solves patients' predicaments of taking medicine and being injected with insulin for long term.

In order to achieve the above purpose of the invention, the present invention adopts the following technical solutions.

Mesenchymal stem cell injection comprises the following components: mescenchymal stem cells of the quantity of 2×10⁵−1×10⁷/ml, human albumin with 1-5% of mass volume ratio, low molecular weight heparin calcium with 0.5% of mass volume ratio, compound amino acid with 1-20% of mass volume ratio, and vitamin C with 0.3-0.7% of mass volume ratio, and the balance is solution medium.

The above technical solution is further improved as follows: the solution medium is compound electrolyte solution, glucose or physiological saline.

The above technical solution is further improved as follows: the mescenchymal stem cells derive from human umbilical cord and/or human placenta, and viability of the stem cells remains at more than 85%.

The present invention further provides a preparation method of the mesenchymal stem cell injection, comprising: preparation of umbilical cord mesenchymal stem cell and preparation of placenta mesenchymal stem cell.

The present invention further provides an application of the mesenchymal stem cell injection in preparing diabetes mellitus drug.

The above technical solution is further improved as follows: the diabetes mellitus comprises Type 1 and Type 2 diabetes mellitus.

The above technical solution is further improved as follows: the prepared mesenchymal stem cell injection is used up within 1 week.

Compared to the prior art, advantages and positive effects of the present invention are: the mesenchymal stem cells adopted by the present invention derive from human umbilical cord and human placenta, and output of the mescenchymal stem cells derived from umbilical cord and placenta is in large quantities, and the preparation system is easy to be subject to Quality Control and industrialization. The components of the mesenchymal stem cell injection are constituted of human mescenchymal stem cells, human albumin, low molecular weight heparin calcium, compound amino acid, vitamin C of 0.5% and solution medium which can be plasma Lyte (compound electrolyte solution) or glucose or physiological saline. In the present invention, human mesenchymal stem cell injection is adopted to repair the damaged islet β cells, restore the functions of diabetes mellitus patient's islet, and lower blood glucose relying on endogenous insulin secretion, so as to fundamentally achieve the purpose of curing diabetes mellitus. The present invention can reverse the course of diabetes mellitus, so that patients can avoid of inconvenience and toxic and side effects due to taking external medicine and daily injection of insulin, and those severe complications caused by poor blood glucose control, therefore improving quality of life of those who have diabetes mellitus.

After reading specific embodiments of the present invention with reference to accompanying drawings, other features and advantages of this invention will become clearer.

### BRIEF DESCRIPTION OF THE DRAWINGS

FIG. 1 is a variation diagram of immune cells in bodies of three groups of mice of the present invention.

FIG. 2 is a comparison diagram between fasting blood glucose and postprandial blood glucose of the three groups of mice of the present invention.

FIG. 3 is a comparison diagram of islet pathological sections of the three groups of mice of the present invention.

FIG. 4 is partial streaming detection results of cells of the present invention.

FIG. 5 is photos of primary cells on the 9th and 13th days of the present invention.

FIG. 6 is photos of 6th generation cells on the 1st and 4th days of the present invention.

### DETAILED DESCRIPTION OF THE PREFERRED EMBODIMENTS

Hereafter the technical solutions of the present invention will be explained in detail in conjunction with the drawings and specific embodiments.

### Embodiment 1

### I. Preparation of Umbilical Cord Mesenchymal Stem Cell

1. Selecting fresh umbilical cord of a mature healthy fetus and rinsing it with PBS buffer liquid which contains penicillin of 100 kU/L and streptomysin of 100 mg/L;

2. Cutting off the umbilical cord with length of 6-8 cm which is cut into segments of length of 2 cm, and repeatedly rinsing the segments with the PBS buffer liquid;

3. Shredding umbilical cord tissue segments into small pieces of volume of 2-3 mm³;

4. Adding the shredded tissue into L-DMEM culture medium and rinsing, centrifuging them for 5 minutes under condition of 500-700 g, and discarding supernatant;

5. Adding culture medium to the tissue pieces at the volume ratio of 2.5-3:1, mixing the tissue pieces evenly, inoculating them into a cell culture dish, then putting the dish in an incubator for cultivation, wherein the culture medium is L-DMEM culture medium containing basic fibroblast growth factor (bFGF) of 1-10 ng/ml and fetal bovine serum (FBS) of the volume percentage of 10%-15%;

6. Refilling the culture medium as Step 5 after 24 hours;

7. Changing the culture medium every 3 days. On the 6th day, changing it with L-DMEM culture medium containing fetal bovine serum of volume percentage of 10%, the cells undergoing passages when reaching about 80% of merge on the 8-9th day, wherein the passage culture medium is the L-DMEM culture medium containing fetal bovine serum of the volume percentage of 10%; and

8. Afterwards, undergoing the passages once every 3 days.

### II. Preparation of Placenta Mesenchymal Stem Cell

1. Rinsing placenta with PBS buffer liquid containing penicillin of 100 kU/L and streptomysin of 100 mg/L, and peeling off amnion on the fetus side slowly along edge of the placental;

2. Again rinsing the amnion from placenta source and cutting the amnion into small pieces of volume of 1-5 mm³;

3. Mixing amnion tissue pieces evenly in DMEM culture medium containing penicillin and streptomysin, and centrifuging it for 10 minutes under condition of 850 g;

4. Discarding supernatant, adding DMEM culture medium containing trypsin of volume percentage of 0.25% in each tube, digesting for 10 minutes at 37° C., and centrifuging it for 10 minutes under condition of 700-900 g;

5. Discarding supernatant, and centrifuging it for 10 minutes under condition of 2200 rpm after adding complete culture medium in each tube, wherein the complete culture medium is DMEM culture medium containing fetal bovine serum of volume percentage of 10%, penicillin of 100 kU/L and streptomysin of 100 mg/L;

6. Discarding supernatant, inoculating the amnion tissue pieces into a cell culture dish, adding the complete culture medium and cultivating it in an incubator, wherein half amount of liquid is changed every 3 days;

7. Cell clones can be observed on the 10-12th days, discarding the tissue pieces after formation of cell clones, adding the complete cultivate medium for cultivation; and

8. Cells cloning and merging to 80-90% on the 15-17th days, and undergoing passages.

### III. Preparation of Mesenchymal Stem Cell Injection

Mesenchymal stem cell injection solution comprises the following components:

the mescenchymal stem cells at the quantity of 2×10⁵−1^{×}10⁷/ml;

clinical grade human albumin with 1-5% of mass volume ratio;

clinical grade low molecular weight heparin calcium with 0.5% of mass volume ratio;

clinical grade compound amino acid with 1-20% of mass volume ratio; and

clinical grade vitamin C with 0.3-0.7% of mass volume ratio, and

the balance is clinical grade solution medium.

For example, stem cell injection of 100 ml is prepared, the injection composed of 5 ml of human albumin solution (final concentration of mass volume ratio of 1%), 0.5 ml of low molecular weight heparin calcium (final concentration of mass volume ratio of 0.5%), 1 ml of compound amino acid (final concentration of mass volume ratio of 1%), 0.5 g of vitamin C (final concentration of mass volume ratio of 0.5%), 93 ml of plasma Lyte (compound electrolyte solution) and mesenchymal stem cells. Except the mesenchymal stem cells, other components of the injection need be prepared in advance, pre-cooled at 4° C. to be used, and the mescenchymal stem cells are finally resuspended in this solution to form single cell suspension, wherein the quantity of the mescenchymal stem cells per milliliter of injection is 2×10⁵. The mescenchymal stem cells still remain in single cell suspension state within 48 hours at the environment temperature of 2-15° C., and viability of the cells (viability of Trypan Blue staining meter) remains at more than 85%. The mass volume ratio of the present invention represents ratio of mass (g) and volume (ml).

The injection enables the mescenchymal stem cells to still remain in single cell suspension state within 48 hours at the temperature of 2-15° C., and viability of the cells remains at more than 85%.

Both human albumin and compound amino acid are components of clinical injection, and can provide nutrition for the cells, being beneficial to metabolism of the cells. The addition of vitamin C can maintain activity of various peroxidases, and is also beneficial to metabolism of the cells and maintenance of the activity.

The addition of 0.5% low molecular weight heparin guarantees that the cells maintain good dispersity state during preservation process, reduces the phenomenon of intercellular agglomerating and the cells adhesive to vessel wall, decreases the danger of possible agglomerating and embolism of cells in blood vessel when clinical cells being infused, while reduces cells loss caused by cells aggregation during being filtered by infusion filter, and the addition of microdosage heparin cannot cause bad reactions such as clinical hemorrhage.

The solution medium, which is plasma Lyte (compound electrolyte solution) or glucose or physiological saline, can maintain osmotic pressure of the cells, being beneficial to survival of the cells. For the components of the injection, it is possible to conveniently select various conventional clinical infusion liquid as solution medium, with compound electrolyte solution as the best.

This injection is very beneficial to survival of the mescenchymal stem cells, and is safe for clinical infusion. The cells can keep higher viability for a long time in this preserved liquid, convenient to clinical transportation without harshness limitation on time, solving the problem of long-time transportation of the cells when used by offsite patients.

### IV. Security and the Validity Experiment of Mescenchymal Stem Cell Injection Treating the NOD Mice (Model of Type 1 Diabetes Mellitus)

In this experiment, 60 female NOD mice of 8 weeks old are selected and are divided into 3 groups at random, 20 per group, which are the comparative group, the preventive group and the treatment group respectively. The comparative group is administrated with physiological saline through tail intravenous injection. At the same time, the preventive group is administrated with mescenchymal stem cell injection of 1 ml (containing mescenchymal stem cells 1.0×10⁶) through tail intravenous injection. After the mice in the treatment group are sickened (the sickened mice are measured as value of fasting blood glucose 11.1 mmol/L, for continuous 2 times), the mescenchymal stem cell injection of 1 ml (containing. 1.0×10⁶ mescenchymal stem cells) through tail intravenous injection. The changes of blood glucose of the mice are monitored every day. After observed for three months, the animals are put to death, blood (about 1 ml) is extracted from the heart and islet tissue pathological sections are subject to relevant experimental check.

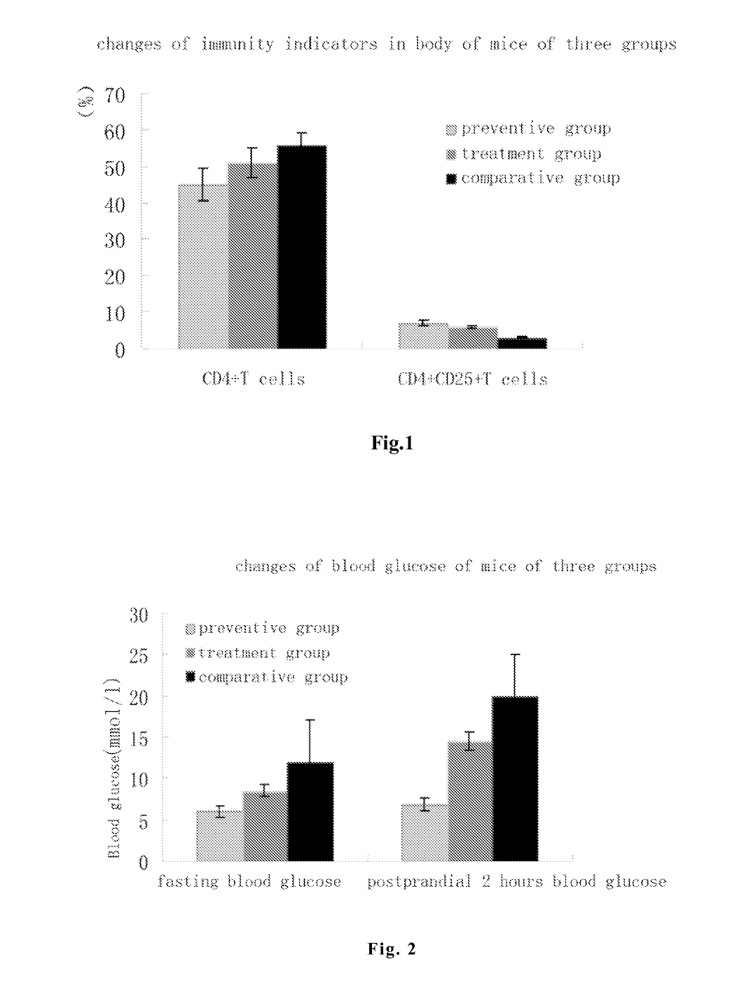

Results show that the intravenous injection of the mescenchymal stem cell injection does not cause acute toxicological response and rejection. The disease time of the mice in the preventive group is obviously later than that of the comparative group, and the disease incidence is obviously lower than that of the comparative group. Mescenchymal stem cell injection can regulate immunologic derangement in the body of the NOD mice, increase the proportion of Treg cells, and reduce the effect of effector T cells, as shown in FIG. 1. Compared with the comparative group, the proportion of the Treg cells in the preventive group and the treatment group is obviously increased, and the proportion of the effector T cells is obviously decreased, P<0.05.

Fasting blood glucose and postprandial blood glucose of the mice in the treatment group and the preventive group are obviously lower than those of the comparative group, as shown in FIG. 2. Compared with the comparative group, fasting blood glucose and postprandial blood glucose of the mice in the preventive group and the treatment group is obviously reduced, P<0.05.

Islet pathological sections show that: function of islet β cells of the mice in the treatment group is obviously recovered compared with the comparative group and the amount of insulin secretion is obviously increased, as shown in FIG. 3. Compared with the comparative group, function of islet β cells of the mice in the treatment group is obviously recovered, and the amount of insulin secretion is obviously increased, P<0.05.

### Embodiment 2

Preparations of umbilical cord and placenta mesenchymal stem cells are the same as those in Embodiment 1.

For example, stem cell injection of 100 ml is prepared, wherein the injection is composed of 25 ml of human albumin solution (final concentration of mass volume ratio being 5%), 0.5 ml of low molecular weight heparin calcium (final concentration of mass volume ratio being 0.5%), 20 ml of compound amino acid (final concentration of mass volume ratio being 20%), 0.5 g of vitamin C (final concentration of mass volume ratio being 0.5%), 54 ml of 5% glucose injection and mesenchymal stem cells. Besides the mesenchymal stem cells, other components of the injection need to be prepared in advance, pre-cooled at 4° C. to be ready for use, and the mescenchymal stem cells are finally resuspended in this solution to form single cell suspension, wherein the quantity of the mescenchymal stem cells per milliliter of injection is 1×10⁷. The mescenchymal stem cells still remain single cell suspension state within 48 hours at the environment temperature of 2-15° C., and viability of the cells remains at more than 85%.

### Embodiment 3

Preparations of umbilical cord and placenta mesenchymal stem cells are the same as those in Embodiment 1.

For example, stem cell injection of 100 ml is prepared, wherein the injection is composed of 10 ml of human albumin solution (final concentration of mass volume ratio being 2%), 0.5 ml of low molecular weight heparin calcium (final concentration of mass volume ratio being 0.5%), 10 ml of compound amino acid (final concentration of mass volume ratio being 10%), 0.5 g of vitamin C (final concentration of mass volume ratio being 0.5%), 79 ml of 0.9% of physiological saline injection and mesenchymal stem cells. Except for the mesenchymal stem cells, other components of the injection need to be prepared in advance, pre-cooled at 4° C. to be ready for use, and the prepared injection is used up within 1 week. The mescenchymal stem cells are finally resuspended in this solution to form single cell suspension, wherein the quantity of the mescenchymal stem cells per milliliter of injection is 2×10⁶. The mescenchymal stem cells still remain single cell suspension state within 48 hours at the environment temperature of 2-15° C., and viability of the cells remains at more than 85%.

### Embodiment 4

### Cultivation and Quality Control of Umbilical Cord Mesenchymal Stem Cells

The umbilical cord mesenchymal stem cells prepared in Embodiment 1 are used to make amplification. Cultivation and amplification of the cells are inoculated at the density of 1.0−1.2×10⁴/cm², complete serum-free culture medium is added, when the cells confluence reaches 80-90%, 0.05% trypsin (not containing EDTA) is used to digest cells, at room temperature. The cells cannot be merged in this digestion solution excessively, otherwise not only the growing contact inhibition can occur, but also stem cells can be brought to spontaneously differentiate, seriously affecting cells growth state after passage.

Flow cytometry detection of immunophenotypes of umbilical cord mesenchymal stem cells

P1 and P6 cells with number of 1×10⁶ are collected respectively, mouse anti human PE-IgG1 and FITC-IgG1 are added to perform homotype contrast, PE and FITC are added to mark mouse anti-human antibody, and immunophenotypes such as CD34, CD45 (marker of hematopoietic cell), CD31 (marker of endothelial cell specific antigen), CD14 (marker of mononuclear macrophage surface), CD90, CD44, CD105 (marker of mesenchyma antigen), HLA-DR (antigen related to transplantation immunity repulsion) are detected.

Cultivation Results and Detection of Cells

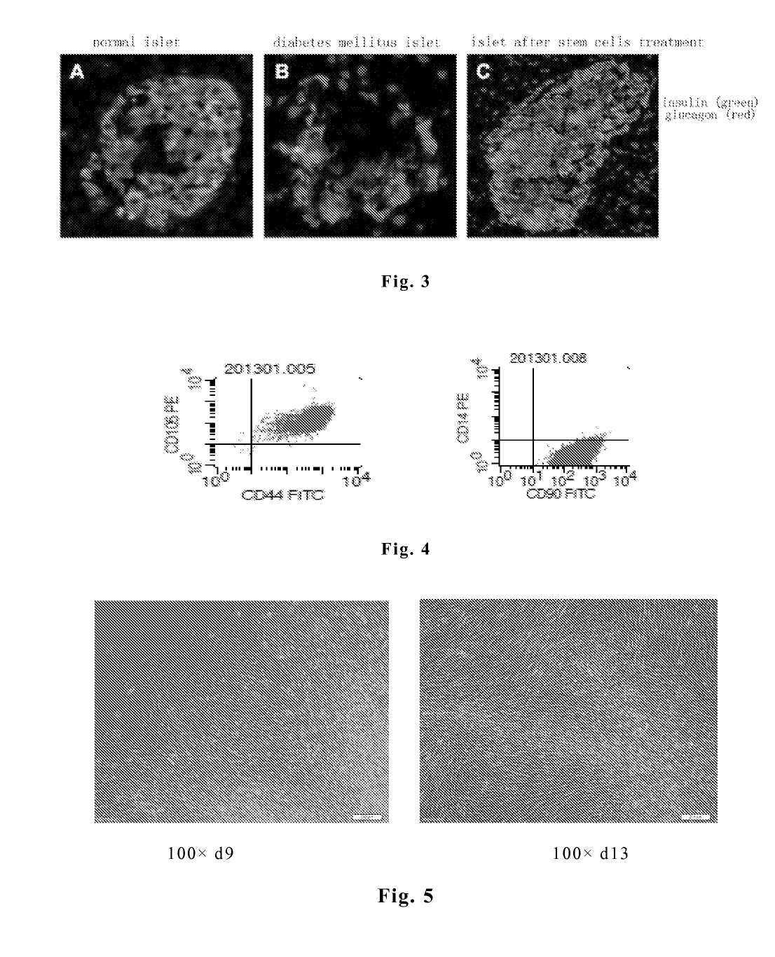

Cells grow against walls, being observed under microscope, and all the forms should be of fusiform, with high diopter. The cells are distributed evenly and aligned uniformly, in whirlpool shape. Acquisition number of primary cells≧1×10⁷; Survival rate of cells (Trypan Blue staining): survival rate of cells before being frozen is 90%, survival rate of cells after being frozen is 85%; the 6th generation cells by continuous passages are of stable forms and are fusiform, distributed evenly and aligned uniformly. Proliferation rate of the 6th generation cells by continuous passage is stable. All phenotypes meet MSC identification standard (CD73, CD105, CD44 or CD90 are positive, and positive rate is not less than 95%; CD31, CD34, CD45, HLA-DR are negative, and positive rate should not be higher than 2%). In detection of cell cycle: 70-80% of the cells are in G0, G1 cell cycle; Both P1 and P6 cells have ability of multi-directional differentiation. Chromosome karyotype analysis is not abnormal. The sum of cells by being amplified continuously to 5-6 generation can reach 10¹⁰˜10¹¹. Partial flow cytometry detection results are shown in FIG. 4.

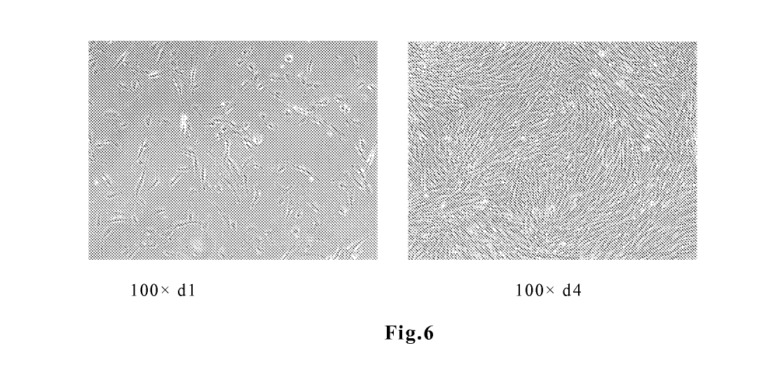

In the above, average cultivated days of primary cells are 13 days, acquisition number of the cells can reach 1.6×10⁷, cells growth state after continuous passage to the 6th generation is good, the cells are all of homogeneous small fusiform, aligned uniformly with whirlpool shape, primary cells are shown in FIG. 5 (photos of the 9th and 13th days), the 6th generation cells are shown in FIG. 6 (photos of the 1st and 4th days).

### Embodiment 5

Umbilical cord mesenchymal stem cell injection (the 3rd generation) is prepared according to Embodiment 2. This injection is used to human patients with diabetes mellitus. 52 Type 2 diabetic patients with disease history less than 5 years are selected. Based on the treatment of diabetic diet control and oral metformin (1500 mg/day) and Avandia (4 mg/day), the examples are divided at random into the treatment group of mescenchymal stem cell and control group, 32 patients in the treatment group and 30 patients in the control group. The patients in treatment group accept infusion of mesenchymal stem cells derived from umbilical cord (2×10⁷ cells per 50 ml), which are injected into pancreas back artery through conduit. Following up to half a year, 1 year, 2 years, results are as follows:

(I) Follow-up indicator changes of Type 2 diabetes mellitus

(1) Base line data comparison: disease course, age, fasting blood glucose, postprandial blood glucose, glycosylated hemoglobin, fasting C-peptide, postprandial half an hour C-peptide, postprandial 1 hour C-peptide, postprandial 2 hours C-peptide of the two groups have no significant differences, each p>0.05);

(2) Islet function of the treatment group increases gradually, and fasting C-peptide, postprandial half an hour C-peptide, postprandial 1 hour C-peptide, and postprandial 2 hours C-peptide reach the peak after 1 year, and are apparently higher than the comparative group (each p<0.05); There is decrease trend as of 2 years, while islet function of the comparative group declines gradually.

C-peptide difference between pretherapy and posttherapy and HbAlc value of the two groups after treatment

| | | |
|---|---|---|
| indicators | treatment group | comparative group |

fasting C-peptide difference (pmol/L)

| | | |
|---|---|---|
| 6 months | 0.14 ± 0.34 | 0.41 ± 0.79 |
| 12 months | 1.62 ± 1.16 | 0.53 ± 0.89 |
| 24 months | 0.26 ± 0.54 | −0.21 ± 0.47 |

postprandial half an hour C-peptide difference (pmol/L)

| | | |
|---|---|---|
| 6 months | 0.32 ± 0.33 | 0.75 ± 1.00 |
| 12 months | 0.85 ± 0.58 | 0.46 ± 0.65 |
| 24 months | 0.81 ± 0.87 | −0.23 ± 1.35 |

Postprandial 1 hour C-peptide difference (pmol/L)

| | | |
|---|---|---|
| 6 months | 0.91 ± 0.49 | 0.66 ± 1.08 |
| 12 months | 0.99 ± 0.77 | 0.53 ± 0.99 |
| 24 months | 0.79 ± 0.44 | −0.41 ± 0.30 |

Postprandial 2 hours C-peptide difference (pmol/L)

| | | |
|---|---|---|
| 6 months | 1.16 ± 1.16 | 0.55 ± 0.89 |
| 12 months | 1.52 ± 1.07 | 0.53 ± 1.06 |
| 24 months | 1.13 ± 0.92 | −0.49 ± 0.90 |

level of HbAlC

| | | |
|---|---|---|
| base line | 7.95 ± 0.64 | 7.87 ± 0.71 |
| 6 months | 6.70 ± 0.70 | 7.3 ± 0.71 |
| 12 months | 6.05 ± 0.33 | 7.35 ± 1.20 |
| 24 months | 6.50 ± 0.57 | 7.57 ± 0.34 |

(II) Follow-up indicator of mesenchymal stem cell derived from umbilical cord changes when treating initial Type 1 diabetes mellitus

13 patients with initial Type 1 diabetes mellitus are selected, and mesenchymal stem cell derived from umbilical cord (2×10⁷) is given through intravenous injection. Following up to 3 months, function of islet β cells and changes of blood glucose and glycosylated hemoglobin (HbAlc) are observed.

Based on insulin treatment, these 13 patients are given mesenchymal stem cell derived from umbilical cord through intravenous injection, and afterwards, following up to 3 months, results show that insulin dosage of 3 patients decreased from 23 u/day to 6 u/day and 2 persons stop using insulin.

| | before | after 3 months of |
|---|---|---|
| indicators | treatment | treatment |
| fasting C-peptide (pmol/L) | 0.20 ± 0.04 | 0.90 ± 0.16 |
| postprandial 1 hour C-peptide | 0.52 ± 0.06 | 2.36 ± 0.68 |
| (pmol/L) | | |
| postprandial 2 hours C-peptide | 0.06 ± 0.05 | 2.09 ± 0.85 |
| (pmol/L) | | |
| HbAlc (%) | 9.3 ± 1.52 | 7.05 ± 0.60 |

Conclusion: mesenchymal stem cells derived from umbilical cord can delay exhaustion of function of islet β cells of Type 2 diabetes mellitus; Mesenchymal stem cells derived from umbilical cord can also delay rapid exhaustion of islet β cells of initial Type 1 diabetes mellitus, reducing insulin dosage, enhancing quality of islet β cells for long-term treatment of Type 1 diabetes mellitus, and then delaying generation and development of diabetic complications.

The above embodiments are only to illustrate the technical solutions of the present invention, but not limit the same. Although the present invention has been described in detail with reference to previous embodiments, for an ordinary person skilled in the art, the technical solutions that are recorded in the embodiments mentioned above may be modified, or partial technical features may be replaced equally. These modifications or replacements would not make the essence of corresponding technical solutions depart from the spirit and scope of the technical solutions required for protection by the present invention.

1. A mesenchymal stem cell injection, comprising:
mescenchymal stem cells in a quantity of 2×10⁵-1×10⁷/ml,
clinical grade human albumin with a mass volume ratio of 1-5%,
clinical grade low molecular weight heparin calcium with a mass volume ratio of 0.5%,
clinical grade compound amino acid with a mass volume ratio of 1-20%, and
clinical grade vitamin C with a mass volume ratio of 0.3-0.7%, and a balance of the injection being a solution medium.

2. The mesenchymal stem cell injection according to claim 1, wherein the solution medium is one of a compound electrolyte solution, glucose or physiological saline.

3. The mesenchymal stem cell injection according to claim 1, wherein the mescenchymal stem cells derive from a human umbilical cord and/or human placenta, and viability of the stem cells remains at more than 85%.

4. A preparation method of the mesenchymal stem cell injection according to claim 3, wherein the human albumin with the mass volume ratio of 1-5%, the low molecular weight heparin calcium with the mass volume ratio of 0.5%, the compound amino acid with the mass volume ratio of 1-20%, the vitamin C with the mass volume ratio of 0.3-0.7% and the solution medium are prepared in advance to form a solution, and then the mescenchymal stem cells are resuspended in the above solution to form a single cell suspension, wherein the quantity of the stem cells is 2×10⁵-1×10⁷/ml.

5. The preparation method of the mesenchymal stem cell injection according to claim 4, wherein preparation of umbilical cord mesenchymal stem cell comprises the following steps:
(1) selecting a fresh umbilical cord of a mature healthy fetus and rinsing it with a PBS buffer liquid which contains penicillin of 100 kU/L and streptomysin of 100 mg/L;
(2) cutting off the umbilical cord with a length of 6-8 cm, and cutting it into segments with lengths of 2 cm, and repeatedly rinsing the segments with the PBS buffer liquid;
(3) shredding umbilical cord tissue segments into small tissue pieces with volume of 2-3 mm³;
(4) adding the shredded tissue pieces into a L-DMEM culture medium and rinsing, centrifuging the culture medium for 5 minutes under a condition of 500-700 g, and discarding supernatant;
(5) adding the tissue pieces into the culture medium according to a volume ratio of 2.5-3:1, mixing the tissue pieces evenly, inoculating culture medium into a cell culture dish, putting the same in an incubator for cultivation; wherein the culture medium is the L-DMEM culture medium containing a basic fibroblast growth factor (bFGF) of 1-10 ng/ml and a fetal bovine serum (FBS) with volume percentage of 10%-15%;
(6) refilling the culture medium in Step 5 after 24 hours;
(7) changing the culture medium every 3 days, changing it on the 6th day with the L-DMEM culture medium containing the fetal bovine serum of volume percentage of 10%, undergoing a passage when up to 80% of the cells merge on the 8-9th days, wherein the culture medium of the passage is the L-DMEM culture medium containing fetal bovine serum with volume percentage of 10%; and
(8) afterwards, undergoing the passage once every 3 days.

6. The preparation method of the mesenchymal stem cell injection according to claim 4, wherein preparation of placenta mesenchymal stem cell comprises the following steps:
(1) rinsing the placenta with the PBS buffer liquid containing penicillin of 100 kU/L and streptomysin of 100 mg/L, and peeling off amnion on a fetus side slowly-along an edge of the placental;
(2) again rinsing an amnion from the placenta source and cutting the amnion into small pieces with volume of 1-5 mm³;
(3) mixing amnion tissue pieces evenly in a DMEM culture medium containing penicillin and streptomysin, and centrifuging the culture medium for 10 minutes under a condition of 850 g;
(4) discarding supernatant, adding the DMEM culture medium containing trypsin with a volume percentage of 0.25% in each tube, digesting for 10 minutes at 37° C., and centrifuging the culture medium for 10 minutes under a condition of 700-900 g;
(5) discarding supernatant, and centrifuging the culture medium for 10 minutes under a condition of 2200 rpm after adding a complete culture medium in each tube, wherein the complete culture medium is DMEM culture medium containing fetal-bovine serum with a volume percentage of 10%, penicillin of 100 kU/L and streptomysin of 100 mg/L;
(6) discarding supernatant, inoculating the amnion tissue pieces into a cell culture dish, adding the complete culture medium and cultivating in an incubator, wherein a half amount of liquid is changed every 3 days;
(7) cells growing against walls on the 10-12th days, discarding the tissue pieces after formation of cell clones, adding the complete cultivate medium and cultivating; and
(8) cell clones merging to 80-90% on the 15-17th days, and making cell passage.

7. Application of the mesenchymal stem cell injection according to claim 1 in preparing a diabetes mellitus drug.

8. The application of the mesenchymal stem cell injection in preparing diabetes mellitus drug according to claim 7, wherein the diabetes mellitus comprises Type 1 and Type 2 diabetes mellitus.

9. The application of the mesenchymal stem cell injection in preparing diabetes mellitus drug according to claim 7, wherein the prepared mesenchymal stem cell injection is used up within 1 week.
